(19) United States
(12) Patent Application Publication
(10) Pub. No.: US 2009/0011012 A1
(43) Pub. Date: Jan. 8, 2009

(54) FATTY ACID COMPOSITIONS AND METHODS OF USE

(76) Inventor: Seth J. Baum, Boca Raton, FL (US)

Correspondence Address: MORRISS OBRYANT COMPAGNI, P.C., 734 EAST 200 SOUTH, SALT LAKE CITY, UT, 84102, US

(21) Appl. No.: 12/167,569

(22) Filed: Jul. 3, 2008

Related U.S. Application Data
(60) Provisional application No. 60/958,613, filed on Jul. 6, 2007.

(51) Int. Cl. A61K 9/48; A61K 31/201; A61K 36/53

(52) U.S. Cl. 424/463; 514/560; 424/725

(57) ABSTRACT

The invention relates to highly concentrated DHA and EPA formulations in a soft gel capsule. A capsule may contain at least 80% omega-3 fatty acids, salts or derivatives thereof, where EPA and DHA are present in relative amounts of greater than or equal to 3:1 or less than or equal to 1:3, and constitute at least 75% to greater than 95% of the total fatty acids present in the capsule. Capsules of the invention may be provided in a blister package so as to provide clean and protected oils that are easy to travel with. Compliance is improved with one-pill-a-day dosing and the days of the week imprinted on the foil packing. Anitoxidant protection may be provided by rosemary and vitamin C. The invention also provides a methods of treatment, modulation or prophalaxis of coronary disease, altering serum LDL-cholesterol and/or HDL-cholesterol, lowering serum triglycerides, lowering blood pressure, pulse rate, altering the activity of the blood coagulation factor VII complex, mild hypertension, protection from cyclosporine toxicity in kidney transplant, rheumatoid arthritis, development and progression of retinopathy, hypertriglyceridemia, and neurological disorders in a subject.

### CROSS-REFERENCE TO RELATED APPLICATIONS

This application claims the benefit of U.S. Provisonal Application No. 60/958,613, filed Jul. 6, 2007, the entirety of which is incorporated by reference.

### TECHNICAL FIELD

Present invention relates to a fatty acid composition comprising omega-3 (all-Z)-5,8,11,14,17-eicosapentaenoic acid (EPA) C 20:5 and (all-Z)-4,7,10,13,16,1 9-docosahexaenoic acid (DHA) C 22:6.

### BACKGROUNDS

Recently, dietary fish oil preparations containing omega-3 polyunsaturated fatty acids have been found, or reported, to reduce triglyceride levels, increase HDL cholesterol levels, reduce homocysteine levels, reduce blood pressure, and/or enhance the effectiveness of statin drugs used to treat cholesterol levels, see U.S. Pat. Nos. 3,082,228; 4,097,602; and 5,698,594; British Patent 2,197,199; and Internation Patent Publication WO 87/02247. For example, consumption of omega 3 fatty acids may be administered to a subject to slow the progression of atherosclerosis and reduce the risk associated with cardiac arrythmias.

In addition, omega-3 fatty acids (EPA and DHA) have been used for the treatment and/or prophalaxis of inflammatory diseases, such as rheumatoid arthritis (especially in early stages of the disease), menstrual cramps, inflammatory bowel disease (ulcerative colitis and Crohn's disease), lupus, and IgA nephropathy, mental or cognitive impairments, such for the treatment of depression, bipolar disorder, schizophrenia, attention deficit disorder, borderline personality disorder, dyslexia and other cognitive impairments, asthma, Raynaud's phenomenon, chronic fatigue syndrome, cystic fibrosis, osteoporosis, prostate cancer, and may also reduce the risk of premature delivery in pregnant women.

Omega-3 fatty acids are also given to pets or other valued animals to help maintain their coats and skin.

In addition, the U.S. Food and Drug Administration (FDA) has issued a qualified health claim for the use of eicosapentaenoic acid (EPA) and docosahexaenoic acid (DHA) omega-3 fatty acids in reducing the risk of coronary heart disease (CHD). This health claim is based on the FDA's finding that credible scientific evidence indicates that these omega-3 fatty acids may be beneficial in reducing CHD.

However, omega-3 fatty acids are subject to spoilage and may contain high levels of undesirable products, such as mercury. Further, ingestion of omega-3 fatty acids frequently results in an undesirable aftertaste or reflux, and the intake of appropriate quantities of the active ingredients—EPA/DHA—often requires ingestion of up to five soft gels daily (a clear impediment to compliance). Therefore, there is a need in the art for a high quality omega-3 supplement, which may be prepared as a highly concentrated and enteric coated capsule.

### SUMMARY OF THE INVENTION

The invention provides the first and only omega-3 fish oil with a full 1,000 mg of DHA+EPA in a soft gel enteric coated capsule that meets the American Heart Association's recommendations for daily supplementation of the omega-3s in patients with cardiovascular disease. In an exemplary embodiment, the invention provides a capsule having the highest amount of vital omega-3 fish oils, DHA and EPA, in a single soft gel capsule that held in a blister package to provide clean and protected oils that are easy to take at home and great to travel with. In another exemplary embodiment, each soft gel in the blister pack is clearly labeled with a day of the week, allowing users to easily self monitor compliance. In addition, an embodiment of the invention provides a DHA to EPA ratio of 3:1. In addition, the invention provides a non-prescription omega-3 supplement that is manufactured in compliance with strict GMP guidelines and is independently assayed for safety and purity.

The invention also providest fatty acid compositions containing a high concentration, at least 80% by weight, of omega-3 fatty acids, salts or derivatives thereof, where EPA and DHA are present in relative amounts of greater than or equal to 3:1 or less than or equal to 1:3, and constitute at least 75% to greater than 95% of the total fatty acids, in a gel capsule having an enteric coating has benefit for the treatment or prophalaxic of cardiovascular and other diseases. One advantage of the compositions according to the invention is their being very well tolerated. Another advantage of the compositions according to the invention is the prolonged shelf-life.

In an exemplary embodiment, the composition according to the invention comprises at least 90% by weight of long chain, polyunsaturated omega-3 fatty acids of which EPA and DHA constitute about 80% by weight of the total fatty acids and are present in a ratio of EPA:DHA from less than or equal to about 1:3 or greater than or equal to about 3:1.

In another exemplary embodiment, the composition according to the invention comprises polyunsaturated omega-3 fatty acids of which EPA and DHA constitute about 82% by weight of the total fatty acids and are present in a ratio of EPA:DHA from less than or equal to about 1:3 or greater than or equal to about 3:1 in a soft gelatin capsule having an enteric coating to prevent reflux in a subject and to improve absorption.

In another exemplary embodiment, the composition according to the invention comprises polyunsaturated omega-3 fatty acids of which EPA and DHA constitute greater than about 80% by weight of the total fatty acids and are present in a ratio of EPA:DHA from less than or equal to about 1:3 or greater than or equal to about 3:1, wherein the composition also contains an antioxidant and the fatty acid and antioxidant are in a soft gelatin capsule having an enteric coating to prevent reflux in a subject.

In another exemplary embodiment, the composition includes vitamin C as an antioxidant. In yet another exemplary embodiment, the composition includes rosemary as an antioxidant, and in yet another exemplary embodiment, the composition includes rosemary and vitamin C as an antioxidant. In yet another exemplary embodiment, the composition of the invention is essentially free of vitamin E (e.g., d-alpha tocopherol), pesticides, chlorinated hydrocarbons, arsenic, cadmium, PCBs, Dioxins, furans, lead and/or mercury.

The invention also provides fatty acid compositions containing an antioxidant and at least 80% by weight omega-3 fatty acids, salts or derivatives thereof, where EPA and DHA are present in relative ratio of about 1:3, and constitute at least 75% to greater than 95% of the total fatty acids present in the composition, wherein the composition is in a gel capsule having an enteric coating. In another exemplary embodiment, the antioxidant is a mixture of rosemary oil and vitamin C. In yet another exemplary embodiment, the antioxidant is a mixture of rosemary extract and vitamin C, wherein the Vitamin C is present in an amount of about 0.3% to about 0.6% of the total fatty acid content, e.g., 5 mg of vitamin C per 1.2 grams of total fat.

In another exemplary embodiment, the composition according to the invention comprises an enteric coated capsule having approximately 60% C22:6 Docosahexacnoic Omega 3 and approximately 20% C20:5 Eicosapentaenoic Omega 3, wherein the total amount of C22:6 and C20:5 is approximately 1,000 mg per capsule. Optionally, each capsule will contain less than about 3% Omega 6 fatty acids, less than about 5% C22:5 Docosahexaenoic Omega 6, less than about 6.5% C22:5 Docosahexaenoic Omega 6, less than about 2% C20:4 Eicosatetraenoic Omega 6, less than about 2.5% C20:4 Eicosatetraenoic Omega 6, ascorbic acid, rosemary extract, and/or the absences of vitamin E. In another exemplary embodiment, the omega 3 fatty acid source is obtained using molecular distillation to remove impurities.

In another exemplary embodiment, the composition according to the invention is packaged as a single approximately 1,000 mg capsule having a 3:1 DHA:EPA ratio where a single capsule is to be consumed once a day and each capsule (i.e., the packaging) is labeled with the day of the week, thereby improving patient compliance and providing a format that can be easily transported by the user. The capsule may be a soft gel capsule, which may be formulated to dissolve in the intestine of the subject, for example, an enteric coated soft gel capsule. The capsules may be packaged in blister packs which are prepackaged cards of a predefined number of blisters, for example, a four column by seven row configuration, where each row represents a day a week and each column represents a different week when medication is to be taken. Alternatively, two approximately three inch by five inch blister packs or sheets of blisters may be used to supply a total of approximately 30 capsules (15 capsules per sheet), for example, using a 3 by 5 matrix of cavities and labeling a first cavity with a day of the week, such as “Sunday.” Each blister is, typically, a clear plastic cavity in deformable plastic base of the blister pack. The blister pack will also have a foil or paper backing material holding the capsule in the cavity, whereby depressing the blister cavity from the top will cause the capsule to puncture through the foil or paper backing so that the capsule is freed from the pack.

In another exemplary embodiment, the composition of the invention includes rosemary oil, vitamin C (e.g., ascorbyl palmitate), gelatin, glycerin, purified water and/or flavorings. In another exemplary embodiment the composition contains no milk, egg, peanut, shellfish, soybean, tree nuts, wheat, yeast, glutten, artificial sweeteners, artificial flavors and/or preservatives.

The invention provides a method of treatment, modulation or prophalaxis of coronary disease (e.g., decreasing the risk of heart attack, abnormal heart rhythums, and strokes), altering serum LDL-cholesterol, LDL-particle number, and/or HDL-cholesterol, lowering serum triglycerides, lowering blood pressure, pulse rate, altering the activity of the blood coagulation factor VII complex, mild hypertension, protection from cyclosporine toxicity in kidney transplant, rheumatoid arthritis, development and progression of retinopathy, hypertriglyceridemia, and neurological disorders, such as Alzheimer's disease, depression, bipolar disorder, attention deficit hyperactivity disorder, schizophrenia, and anxiety disorders in a subject, the method comprising administering an effective amount of a nutritional supplement comprising omega-3 fatty acid, or a derivative thereof (e.g., an ester thereof), to the subject.

### DETAILED DESCRIPTION OF THE INVENTION

As used herein and in the appended claims, “about” means reasonably close to, or approximately, a little more or less than the stated number or amount.

As used herein and in the appended claims, a “Subject” refers to a mammal, including a human, cat, dog, or horse. A subject may also be referred to as a “patient.”

As used herein and in the appended claims, the singular forms “a”, “an”, and “the” include plural reference unless the context clearly dictates otherwise.

As used herein, “comprising,” “including,” “containing,” “characterized by,” and grammatical equivalents thereof are inclusive or open-ended terms that do not exclude additional, unrecited elements or method steps, but also includes the more restrictive terms “consisting of” and “consisting essentially of.”

As used herein, “DHA” means (C22:6 n=3) docosahexaenoic acid or derivatives thereof and “EPA” means (C20:5 n=3) eicosapentaenoic acid or derivatives thereof, where both terms include triglyceride, ester ethyl esters, and/or acid salts thereof.

As used herein, “dosage form” means a unit of administration for a composition of the invention, for example, a tablet, capsule, particularly a gel or liquid capsule, and the like.

As used herein, “effective amount” or “therapeutically effective amount” means an amount effective, when administered to a subject, to provide any therapeutic benefit, including treatment, modulation of an indicia of disease, or prophalaxis.

The invention provides highly purified omega-3 fatty acid formulations and unit dosage forms thereof. The invention also provides methods of using the dosage forms to treat a variety of cardiovascular, autoimmune, inflammatory, central nervous system disorders, or chronic pain by providing or administering a formulation of the invention to a subject.

EPA is used to produce beneficial eicosanoids, which regulate many organ systems, for example; they decrease blood pressure, inflammation, cell proliferation, heart disease and platelet aggregation. The eicosanoids formed from EPA thus provide a protective balance that prevents or delays the onset of many deleterious conditions. In contrast, DHA has less of a role in forming Eicosanoids; however, it is a major constituent of the plasma membrane in neuronal cells of the brain, the retina cells of the eye, and is important for all cell membranes. DHA is the precursor to the Protectins, powerful anti-inflammatory substances having especially important neural-protective activity. In addition to the ability to be converted into eicosanoids, EPA may also be converted into DHA (and to some degree a process of retroconversion may occur as well). However, the conversion rate in many people is probably not sufficient to maintain beneficial levels of DHA.

Low levels of DHA have been associated with neurological and behavioral disorders such as depression, Alzheimer's disease, Attention Deficit Hyperactivity Disorder, and other disorders. Therefore, it is beneficial to provide omega-3 fatty acid with a favorable ratio of DHA to EPA, such that DHA is present at a higher concentration than EPA. In particular, the invention provides a approximately 3:1 ratio of DHA to EPA, since DHA is more biologically active than EPA, is taken up more avidly by membranes, and is present in the brain and macula, while EPA is not. Further, this ratio does not require conversion of EPA to DHA in the subject, thereby making the formulation more beneficial. Providing omega-3 fatty acids with a higher concentration of DHA may be beneficial to some subjects and provides a nutritional formulation that more closely resembles the concentrations of DHA and EPA that would have been found in human diets rich in salmon and other healthful fish.

In an exemplary embodiment, an enteric coated softgel capsule is used to deliver a composition of the invention, thereby inhibiting release of the fish oil prior to clearing the stomach. This is particularly beneficial in reducing the undesirable side effects of upset stomach, aftertaste and reflux, while increasing absorption by releasing the EPA and DHA directly into the small intestine where it can be efficiently absorbed (see U.S. Pat. No. 7,792,795 and International Patent Publication WO 90/04391).

In another exemplary embodiment, the omega-3 fatty acids may be provided in the form of an ester, ethyl ester, triglyceride, free acid or other derivative forms.

Omega-3 fatty acids are particularly subject to spoilage by lipid peroxidation, which may be delayed or prevented by the addition of an effective amount of an antioxidant, wherein an effective amount of an antioxidant may be assayed by measurement of peroxide (which primarily indicates recent spoilage) and anisidine (which primarily indicates longer-term spoilage) levels over time (A. R. Hra\check{s} et al. (2000), Comparison of antioxidative and synergistic effects of rosemary extract with α-tocopherol, ascorbyl palmitate and citric acid in sunflower oil. Food Chemistry 71:229-233). In order to preserve highly concentrated fish oil and/or manufacture the capsules of the invention, it may be desirable to store the oil under a noble gas prior to filling the capsules, e.g., storage under Argon gas so as to prevent oxidation of the fish oil.

In another exemplary embodiment, the composition of the invention comprises an antioxidant (such as catechin, vitamin C, vitamine E, TBHQ, carotenoids, astaxanthin, bioflavonoids and/or natural antioxidants), for example, in an amount between about 0.001% and about 0.1%, about 0.005% to about 0.05%, or about 0.01% and about 0.05% or about 0.03%, by weight. Exemplary antioxidants are described in U.S. Patent Publication 2005/0192634 and U.S. Pat. No. 5,527,533. In another exemplary embodiment, the antioxidant comprises rosemary, for example, rosemary oleoresin extract. In another exemplary embodiment, the composition comprises rosemary in an amount of about 0.01% to about 0.03%, about 0.005% to about 0.1%, or about 0.001% to about 0.5%, by weight. In yet another exemplary embodiment, the antioxidant comprises vitamin C, for example, ascorbyl palminate. In another exemplary embodiment, the composition comprises vitamin C in an amount of about 0.001% to about 0.1%, about 0.005% to about 0.05%, or about 0.009% to about 0.011%, by weight. (see, U.S. Patent Publications 2007/0141138 and 2005/0184275).

In another exemplary embodiment, the composition of the invention comprises a mixture of vitamin C and rosemary extract. For example, rosemary extract may be present in an amount of about 0.02% and vitamin C (e.g., ascorbyl palminate) may be present in an amount of about 0.4%. In another exemplary embodiment, a rosemary extract may be used in a contentration of about 0.01% to about 5%, and vitamin C in a concentration of about 0.1% to about 1% of the total fat concentration. For example, a capsule containing about 1.2 g of total fat and 5 mg of vitamin C would have a vitamin C concentration of about 0.4%.

In an exemplary embodiment, compositions of the invention include high concentrations of EPA and DHA, where the EPA and DHA comprise at least about 75%, at least about 80%, at least about 82%, at least about 83%, at least about 85%, at least about 90%, by weight, of the total fatty acids.

In an exemplary embodiment, the composition comprises approximately 1,020 mg to approximately 1,239 mg of high quality omega-3 fish oil (about 992 mg or about 1,000 mg of which is EPA and DHA in a ratio of about 1:3), approximately 0.3 mg of rosemary extract, and approximately 0.1 mg vitamin C (ascorbic acid, sodium ascorbate, calcium ascorbate or ascorbyl palmitate), in a capsule having an enteric coating.

The omega-3 fatty acid of the invention may be obtained from any desirable and appropriate source, such as a high quality omega-3 fish oil or a pharmaceutical grade fish oil.

The compositions of the invention may contain excipients, such as, fillers, stabilizers, extenders, binders, humidifiers, surfactants, lubricants, and the like. Excipients are selected with respect to the intended form of administration, e.g. oral tablets, capsules, powders, syrups, suspensions, and the like, and consistent with conventional pharmaceutical practices. For example, for oral administration in the form of gel capsule a composition of the invention may be combined with a flavorant, colorant or the like.

The amounts of omega-3 formulation contained in an oral unit dosage form for adult human patients may be from about 400 mg to about 1400 mg of omega 3 fatty acids, about 700 mg to about 1,300 mg, about 800 mg to about 1,200 mg, about 900 mg to about 1,100 mg, or about 400 mg to about 600 mg. Unit dosage forms for adult human patients may generally contain between about 900 mg to about 1,000 mg of purified EPA and DHA. Unit dosage forms may contain at least about 900 mg, at least about 930 mg, at least about 960 mg, at least about 980 mg, at least about 990 mg, at least about 1,000 mg, or about 1,000 mg of EPA and DHA in a single capsule. Unit dosage forms may also contain at least about 400 mg, at least about 450 mg, at least about 500 mg, or at least about 600 mg of EPA and DHA in a single capsule. Unit dosage forms for pediatric or veterinary use may contain different amounts of Omega-3 fatty acids depending on the subject to be treated. Frequency of dosage may also vary depending on the rout of administration and the particular disease treated, with a dosage regimen of 4 times daily or less generally being sufficient for most diseases (a dosage regimen of 1 or 2 times daily or less being desirable).

It will be understood, however, that the specific dose level for any particular subject will depend upon a variety of factors including the age, body weight, general health, sex, diet, time of administration, route of administration, and the severity of the particular disease undergoing therapy.

A capsule shell may be made of methylcellulose, hydroxypropylmethyl cellulose, polyvinyl alcohols, or denatured gelatins or starch, bone or pork skin gelatins, or other material. Other suitable capsule shell materials include polyethylene, polypropylene, poly(methylmethacrylate), polyvinylchloride, polystyrene, polyurethanes, polytetrafluoroethylene, nylons, polyformaldehydes, polyesters, cellulose acetate, and nitrocellulose. The capsule shell itself may contain small amounts of dyes, opaquing agents, plasticizers, and preservatives. Gelatin capsule shells may be made also be made of tapioca, grass, vegetable derived or fish derived gelatin. In other embodiments the capsule has a shell comprising the material of the rate-limiting membrane, including coating materials, and filled with Omega-3 fatty acids. Capsule shells may be made of a porous or a pH-sensitive polymer made by a thermal forming process. In certain embodiments the capsule shell in the form of an asymmetric membrane; i.e., a membrane that has a thin skin on one surface and most of whose thickness is constituted of a highly permeable porous material.

The dosage forms of the invention may include an enteric coating, which is resistant to digestion in the stomach, but substantially soluble in the small intestine. Examples of coating materials include cellulose, vinyl, and acrylic derivatives, such as polyvinyl acetate phthalate (PVAP), hydroxypropylmethylcellulose acetate succinate (HPMCAS), cellulose acetate phthalate (CAP), methacrylic acid copolymer, hydroxypropylmethylcellulose succinate, cellulose acetate succinate, cellulose acetate hexahydrophthalate, hydroxypropylmethylcellulose hexahydrophthalate, hydroxypropylmethylcellulose phthalate (HPMCP), cellulose propionate phthalate, cellulose acetate maleate, cellulose acetate trimellitate, cellulose acetate butyrate, cellulose acetate propionate, methacrylic acid/methacrylate polymer, methacrylic acid-methyl methacrylate copolymer, ethyl methacrylate-methylmethacrylate-chlorotrimethylammonium ethyl methacrylate copolymer, and combinations thereof.

The composition of the invention may be encapsulated and a predetermined number of capsules packaged in what is typically referred to as a blister pack or push-through pack that provides a further barrier to oxidation, protection from product tampering, and/or a compliance aid by printing the days of the week above each dosage unit. In an exemplary embodiment the blister pack comprises a deformable plastic base into which the dosage unit fits and a backing material, such as foil or paper, through which the dosage units are retrieved by the user.

### EXAMPLE I

An exemplary embodiment comprises an enteric coated soft gelatin capsule having the following properties:

Fill Material

| Ingredient | Quantity in mg |
|---|---|
| Rosemary Oleoresin Extract | 0.24 |
| Fish Oil 200/600 EE | 1239.64 |
| Vitamin C (Ascorbyl Palmitate) | 0.12 |
| Total Fill Weight | 1240.00 |

Where the omega-3 and other fatty acids may comprise:

| | | % of total | Mg per |
|---|---|---|---|
| Common Name | Method | fatty acid | capsule |
| Eicosapentaenoic acid (EPA) | Internal Gas | 21.97 | 247.95 |
| | Chromatography | | |
| Docosahexaenoic acid (DHA) | Internal Gas | 65.91 | 743.74 |
| | Chromatography | | |
| Total Omega-3 Fatty Acid | Internal Gas | 89.97 | 1,015.27 |
| | Chromatography | | |
| d-alpha Tocopherol | Internal Gas | 0.0 | 0.0 |
| | Chromatography | | |

Where the capsules have no detectable Escherichia coli, Pseudomonas aeruginosa, Salmonella, and Staphylococcus aureus, have arsenic, cadmium, lead and mercury levels at or below about 0.1 ppm, PCBs (IUPAC numbers 28, 52, 101, 118, 138, 153 and 180) at or below about 0.09 ppm and dioxins and furans at or below about 2.0 picograms-WHO-TEQ/g.

The composition may be filled in oblong soft gelatine capsules (e g., size 20, average weight 1.4 g) using a standard capsulation machine.

### EXAMPLE II

An exemplary embodiment comprises an enteric coated soft gelatin capsule having the following properties:

Fill Material

| Ingredient | Quantity |
|---|---|
| Rosemary Cleoresin Extract | About 0.24 mg |
| Total Fat | About 1200 mg |
| Vitamin C (Ascorbyl Palmitate) | About 0.12 mg |
| Omega-3 fatty acids | About 1020 mg |
| DHA | About 750 mg |
| EPA | About 250 mg |

Where the capsules have no detectable Escherichia coli, Pseudomonas aeruginosa, Salmonella, and Staphylococcus aureus, have arsenic, cadmium, lead and mercury levels at or below about 0.1 ppm, PCBs (IUPAC numbers 28, 52, 101, 118, 138, 153 and 180) at or below about 0.09 ppm and dioxins and furans at or below about 2.0 picograms-WHO-TEQ/g.

The capsules may be packaged in a blister packaging system comprising a push-through-pack system having 30 capsules per individual sheet as they will be sold to the consumer.

### EXAMPLE III

An exemplary embodiment comprises an enteric coated soft gelatin capsule having the following properties:

Fill Material

| Ingredient | Quantity |
|---|---|
| Rosemary Oleoresin Extract | About 0.20 mg |
| Total Fat | About 1000 mg |
| Vitamin C (Ascorbyl Palmitate) | About 0.10 mg |
| DHA | About 200 mg |
| EPA | About 300 mg |

While this invention has been described in certain embodiments, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

All references, including publications, patents, and patent applications, cited herein are hereby incorporated by reference to the same extent as if each reference were individually and specifically indicated to be incorporated by reference and were set forth in its entirety herein.

What is claimed is:

1. A composition containing a fatty acid composition comprising (all-Z)-5,8,11,14,17-eicosapentaenoic acid (EPA) C 20:5, or derivatives thereof, and (all-Z)-4,7,10,13,16,19-docosahexaenoic acid (DHA) C 22:6, or derivatives thereof, in a ratio of EPA:DHA of less than or equal to about 1:3 or greater than or equal to about 3:1, wherein the EPA and DHA comprise at least 80% of the fatty acids present in the composition and an antioxidant.

2. The composition of claim 1, wherein the antioxidant is present in an amount between about 0.01% to about 0.05% by weight relative to the weight of the fatty acid.

3. The composition of claim 2, wherein the antioxidant is a mixture of vitamin C and rosemary oil.

4. The composition of claim 1, wherein the composition is present in a capsule.

5. The composition of claim 4, wherein the capsule comprises an enteric coated capsule.

6. A nutritional supplement comprising a fatty acid composition having a ratio of C22:6 docosahexaenoic omega 3 fatty acid (DHA) to C20:5 eicosapentaenoic omega 3 fatty acid (EPA) of approximately 3: 1, wherein the EPA and DHA constitute at least about 80% by weight of all fatty acids present in the fatty acid composition and wherein the fatty acid composition comprises rosemary oil and vitamin C as an antioxidant and wherein the fatty acid composition is contained in a capsule.

7. The composition of claim 6, wherein the capsule is an enteric coated capsule.

8. The composition of claim 6, wherein the capsule contains at least 1,000 mg of the fatty acid.

9. The composition of claim 6, wherein the capsule contains a total amount of DHA and EPA of at least 900 mg.

10. The composition of claim 6, wherein the capsule contains a total amount of DHA and EPA of at least 990 mg.

11. The composition of claim 6, wherein the composition comprises less than about 6.5% C22:5 Docosahexaenoic Omega 6.

12. The composition of claim 6, wherein the composition comprises less than about 2.5% C20:4 Eicosatetraenoic Omega 6.

13. The composition of claim 6, wherein the composition is substantially free of a contaminant selected from the group consisting of mercury, lead, arsenic, cadmium, PCBs, dioxins, furans and combinations thereof.

14. The composition of claim 6, wherein at least 83% by weight of the fatty acids comprise long chain, polyunsaturated, omega-3 fatty acids.

15. The composition of claim 6, wherein at least 90% by weight of the fatty acids comprise long chain omega-3 fatty acids.

16. The composition of claim 6, wherein the fatty acids are present in the composition predominantly in an esterified form.

17. The composition of claim 6, wherein the fatty acids are present in the composition predominantly in an ethyl ester form.

18. The composition of claim 6, wherein the fatty acids are present in the composition predominantly in a free acid form.

19. A unit dosage delivery system comprising:
a deformable plastic sheet having a series of cavities, wherein each cavity is configured to receive a single capsule;
a plurality of capsules comprising a fatty acid having a ratio of C22:6 Docosahexaenoic Omega 3 (DHA) to C20:5 Eicosapentaenoic Omega 3 (EPA) of approximately 3:1, wherein the EPA and DHA constitute at least about 80% by weight of all fatty acids present in the capsule, wherein the capsules are present in the series of cavities; and
a backing material adhered to the plastic sheet, wherein the foil backing retains one of the plurality of capsules in each cavity of the plastic sheet and wherein the capsule may be pushed through the foil backing.

20. The unit dosage delivery system of claim 19, wherein the deformable plastic sheet comprises at least five cavities in a row or a column.

21. The unit dosage delivery system of claim 19, wherein the backing material is printed with a day of the week corresponding to each cavity in the row or column.

22. The unit dosage delivery system of claim 19, wherein the deformable plastic sheet comprises a series of thirty cavities.

23. The unit dosage delivery system of claim 19, wherein each capsule comprises approximately 750 mg of DHA and approximately 250 mg of EPA.

24. The unit dosage delivery system of claim 19, wherein at least 83% by weight of the fatty acids comprise long chain, polyunsaturated, omega-3 fatty acids.

25. The unit dosage delivery system of claim 19, further comprising rosemary oil and vitamin C.
